(19) United States
(12) Patent Application Publication
(10) Pub. No.: US 2025/0152123 A1
(43) Pub. Date: May 15, 2025

(54) WEARABLE ULTRASONIC BLOOD PRESSURE MONITOR AND USE THEREOF

(71) Applicant: Salyx Medical Inc., Victoria (CA)

(72) Inventors: Alfonso Fabian de la Fuente Sanchez, Victoria (CA); Sergio Perez Martell, Victoria (CA)

(73) Assignee: Salyx Medical Inc., Victoria (CA)

(21) Appl. No.: 19/012,719

(22) Filed: Jan. 7, 2025

Related U.S. Application Data
(62) Division of application No. 18/324,876, filed on May 26, 2023.

(51) Int. Cl. A61B 8/04; A61B 8/00

(52) U.S. Cl. CPC A61B 8/04; A61B 8/4236; A61B 8/4494; A61B 8/565; A61B 8/461; A61B 2562/164

(57) ABSTRACT

A wearable blood pressure monitoring patch for use with a mobile computing device is provided, the wearable blood pressure monitoring patch comprising: a flexible housing which includes a contact surface; an adhesive on the contact surface; an array of ultrasound transducers embedded in the flexible housing and facing the contact surface; a high-speed analogue to digital converter in electrical communication with the array of ultrasound transducers; and a microprocessor which includes a wireless radio for transmitting ultrasound data to the mobile computing device and is in electronic communication with the high speed analogue to digital converter. A system which includes the mobile computing device is also provided.

### CROSS REFERENCE TO RELATED APPLICATIONS

The present application is a divisional application of, and claims benefit of U.S. patent application Ser. No. 18/324,876 filed May 26, 2023 and entitled WEARABLE ULTRASONIC BLOOD PRESSURE MONITOR AND USE THEREOF, the contents of which are incorporated herein by reference in their entireties.

### FIELD

The present technology is directed to a wearable patch for the continuous monitoring of blood pressure. More specifically it is an ultrasonic blood pressure monitor system which includes an application on a mobile computing device and may include a cloud system for machine learning processing.

### BACKGROUND

U.S. Pat. No. 9,770,177 discloses a ring-type cuff for blood pressure manometer, wherein the cuff (C) comprises: a circular rubber ring (1) wearable on a wrist or an ankle; a resilient coil spring (2) built-in inside the tubular rubber ring (1) in the range of length of the rubber ring to obtain a predetermined flexibility, to suppress excessive stretching, to strengthen durability, and to maintain resilience; and a measurement portion (3) comprising a pressure sensor (S) for measuring blood pressure, a transmission circuit (IC) for transmitting measurement data to the outside, a power source (v), a power switch (sw), and a pilot lamp (LED). The ring-type cuff (C) is worn on the wrist or the ankle at ordinary times; when measuring blood pressure, the ring-type cuff (C) is placed on a measurement site to be fixed by a self-elastic supporting force, blood pressure is measured by the pressure sensor (S) of the measurement portion (3) and the pilot lamp (LED) is simultaneously lighted, the measured data is wired and wirelessly transferred to an external sphygmomanometer by the transmission circuit (IC) to be analyzed, and the analyzed result is displayed on a display portion through a letter or a figure; and the pilot lamp is also turned off if the ring-type cuff (C) descends to the wrist after measurement. While this is a wearable blood pressure monitor, it is bulky and relies on a pressure sensor for measuring blood pressure.

United States Patent Application Publication No. 20050154299 discloses that ultrasound is used to provide input data for a blood pressure estimation scheme. The use of transcutaneous ultrasound provides arterial lumen area and pulse wave velocity information. In addition, ultrasound measurements are taken in such a way that all the data describes a single, uniform arterial segment. Therefore a computed area relates only to the arterial blood volume present. Also, the measured pulse wave velocity is directly related to the mechanical properties of the segment of elastic tube (artery) for which the blood volume is being measured. In a patient monitoring application, the operator of the ultrasound device is eliminated through the use of software that automatically locates the artery in the ultrasound data, e.g., using known edge detection techniques. Autonomous operation of the ultrasound system allows it to report blood pressure and blood flow traces to the clinical users without those users having to interpret an ultrasound image or operate an ultrasound imaging device. This still relies on an inflatable cuff for measuring blood pressure.

What is needed is a small, wearable blood pressure monitor. It would be preferable if it was a patch that could be worn by a user. It will be preferable if the patch had ultrasound transducers embedded therein. It would be preferable if the blood pressure monitor was provided as a system that included an application on a mobile device. It would be preferable if the blood pressure monitor included a wireless radio for communicating ultrasound data to the application. It would also be preferable if the application included a machine learning component.

### SUMMARY

The present technology is a small, wearable blood pressure monitor. It is a patch that can be worn by a user. The patch has ultrasound transducers embedded therein. The blood pressure monitor is provided as a system that includes an application on a mobile device. The blood pressure monitor included a wireless radio for communicating ultrasound data to the application. The application includes a machine learning component.

In one embodiment a blood pressure monitoring system is provided, the blood pressure monitoring system comprising a wearable patch and a mobile computing device, the wearable patch including: a flexible housing which includes a contact surface; an adhesive on the contact surface; an array of ultrasound transducers embedded in the flexible housing and facing the contact surface; a high speed analogue to digital converter in electrical communication with the array of ultrasound transducers; and a microprocessor which includes a wireless radio for transmitting ultrasound data to the mobile computing device and is in electronic communication with the high speed analogue to digital converter, the mobile computing device including: a memory; a processor; a wireless receiver; and a screen, wherein the memory is configured to instruct the processor to convert the ultrasound data into blood pressure data and to display the blood pressure data on the screen.

In the blood pressure monitoring system, the flexible housing may be a silicon housing.

In the blood pressure monitoring system, the memory of the mobile computing device may be configured for learning.

In the blood pressure monitoring system, the high speed analogue to digital converter may be configured to operate at over one million samples per second.

In another embodiment, a wearable blood pressure monitoring patch for use with a mobile computing device is provided, the wearable blood pressure monitoring patch comprising: a flexible housing which includes a contact surface; an adhesive on the contact surface; an array of ultrasound transducers embedded in the flexible housing and facing the contact surface; a high speed analogue to digital converter in electrical communication with the array of ultrasound transducers; and a microprocessor which includes a wireless radio for transmitting ultrasound data to the mobile computing device and is in electronic communication with the high speed analogue to digital converter.

In the wearable blood pressure monitoring patch, the flexible housing may be a silicon housing.

In the wearable blood pressure monitoring patch, the high speed analogue to digital converter may be configured to operate at over one million samples per second.

In another embodiment, a method of monitoring a user's blood pressure is provided, the method comprising: selecting the blood pressure monitoring system described above; the user releasably attaching the wearable patch to a selected skin surface; the ultrasound transducer emitting ultrasound waves under control of the microprocessor; the ultrasound transducer receiving reflected ultrasound waves; the analogue to digital converter digitizing the reflected ultrasound waves to provide a data set; the wireless radio sending the data set to the mobile computing device; and the mobile computing device analyzing the data set to provide a blood pressure reading.

The method may further comprise the mobile computing device displaying the blood pressure reading in real time.

In another embodiment, a blood pressure monitoring system is provided, the blood pressure monitoring system comprising a wearable patch, a mobile computing device and a remote computer, the wearable patch including: a flexible housing which includes a contact surface; an adhesive on the contact surface; an array of ultrasound transducers embedded in the flexible housing and facing the contact surface; a high speed analogue to digital converter in electrical communication with the array of ultrasound transducers; and a microprocessor which includes a wireless radio for transmitting ultrasound data to the mobile computing device and is in electronic communication with the high speed analogue to digital converter, the mobile computing device including: a memory; a processor; a wireless radio; and a screen, wherein the memory is configured to instruct the processor to send the ultrasound data to the remote computer and to display blood pressure data on the screen, and the remote computer including: a memory; a processor; and a wireless radio wherein the memory is configured to convert the ultrasound data into blood pressure data and send the blood pressure data to the mobile computing device.

In the blood pressure monitoring system, the flexible housing may be a silicon housing.

In the blood pressure monitoring system, the memory of the remote computer may be configured for learning.

In the blood pressure monitoring system, the high speed analogue to digital converter may be configured to operate at over one million samples per second.

In another embodiment, a method of monitoring a user's blood pressure, the method comprising: selecting the blood pressure monitoring system described above; the user releasably attaching the wearable patch to a selected skin surface; the ultrasound transducer emitting ultrasound waves under control of the microprocessor; the ultrasound transducer receiving reflected ultrasound waves; the analogue to digital converter digitizing the reflected ultrasound waves to provide a data set; the wireless radio sending the data set to the mobile computing device; the mobile computing device sending the data set to the remote computer; the remote computer analyzing the data set to provide a blood pressure reading; the remote computer sending the blood pressure reading to the mobile computing device; and the mobile computing device displaying the blood pressure reading on the screen.

In the method, the displaying the blood pressure reading may be in real time.

### FIGURES

FIG. 1 is a schematic of the blood pressure monitoring system of the present technology.

FIG. 2 is a schematic of a sectional view of the wearable patch of the system of FIG. 1.

FIG. 3 is a schematic of the machine learning system of the present technology.

FIG. 4 is a flow chart of the learning process.

FIG. 5 is a flow chart of the method of blood pressure monitoring of the present technology.

### DESCRIPTION

Except as otherwise expressly provided, the following rules of interpretation apply to this specification (written description and claims): (a) all words used herein shall be construed to be of such gender or number (singular or plural) as the circumstances require; (b) the singular terms “a”, “an”, and “the”, as used in the specification and the appended claims include plural references unless the context clearly dictates otherwise; (c) the antecedent term “about” applied to a recited range or value denotes an approximation within the deviation in the range or value known or expected in the art from the measurements method; (d) the words “herein”, “hereby”, “hereof”, “hereto”, “hereinbefore”, and “hereinafter”, and words of similar import, refer to this specification in its entirety and not to any particular paragraph, claim or other subdivision, unless otherwise specified; (e) descriptive headings are for convenience only and shall not control or affect the meaning or construction of any part of the specification; and (f) “or” and “any” are not exclusive and “include” and “including” are not limiting. Further, the terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller sub ranges are also included. The upper and lower limits of these smaller ranges are also included therein, subject to any specifically excluded limit in the stated range.

Unless defined otherwise, all technical and scientific terms used herein have the same meaning as commonly understood by one of ordinary skill in the relevant art. Although any methods and materials similar or equivalent to those described herein can also be used, the acceptable methods and materials are now described.

### Definitions

Mobile computing device—in the context of the present technology a mobile computing device is a cellular phone, a smart phone, a tablet, a laptop, wearable computing device, personal digital assistant, mobile Internet device, notebook, netbook, smart device and any computing device that is either hand-held or can easily be held by an individual.

Computing device—in the context of the present technology, a computing device includes at least one processor, a network adapter, and computer-readable storage media. A network adapter may be any suitable hardware and/or software to enable the computing device to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable media may be adapted to store data to be processed and/or instructions to be executed by processor. The processor enables processing of data and execution of instructions. The data and instructions may be stored on the computer-readable storage media.

A computing device may additionally have one or more components and peripherals, including input and output devices. Examples of output devices that can be used to provide a user interface or display screens for visual presentation of output and speakers or other sound generating devices for audible presentation of output.

Computer executable instructions—in the context of the present technology, computer executable instructions include software, including as application software, system software, firmware, middleware, embedded code, or any other suitable type of computer code and also may be compiled as executable machine language code or intermediate code.

### DETAILED DESCRIPTION

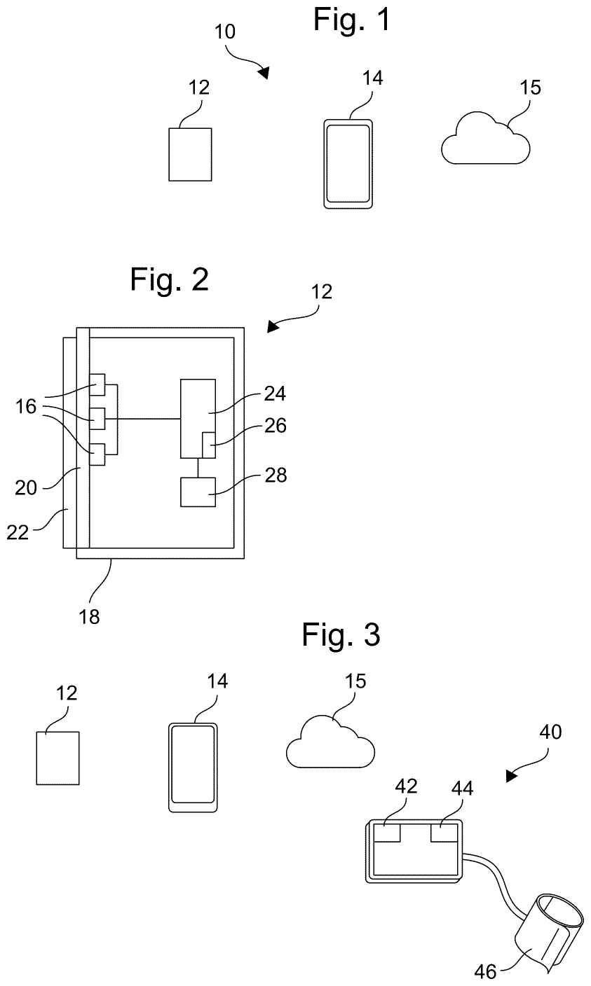

A blood pressure monitoring system, generally referred to as 10 is shown in FIG. 1. The blood pressure monitoring system 10 includes a wearable patch 12 and a mobile computing device 14. In one embodiment, the system 10 includes a remote computer 15 which provides cloud processing.

As shown in FIG. 2, the wearable patch 12 has an array of ultrasound transducers 16 embedded in a flexible housing 18, facing a contact surface 20. In one embodiment the flexible housing 18 is silicon. In another embodiment, the flexible housing is three dimensionally printed plastic polymer. The contact surface 20 is coated, at least partially, with an adhesive layer 22, for affixing the wearable patch 12 to a user. The ultrasound transducers are in electrical communication with at least one microcontroller 24. The microcontroller 24 includes a wireless radio 26. A high-speed analogue to digital converter 28 is in electronic communication with the microcontroller 24 or may be integrated in the microcontroller 24. The high-speed analogue to digital converter 28 preferably operates at over one million samples per second. A battery is in electrical communication with the ultrasound transducers 16, the microcontroller 24 and the high-speed analogue to digital converter 28.

As shown in FIG. 3, a machine learning system includes the wearable patch 12, the mobile computing device 14, remote computer 15 and a sphygmomanometer, generally referred to as 40, which includes an analogue to digital converter 42, a wireless radio 44 and an inflatable cuff 46.

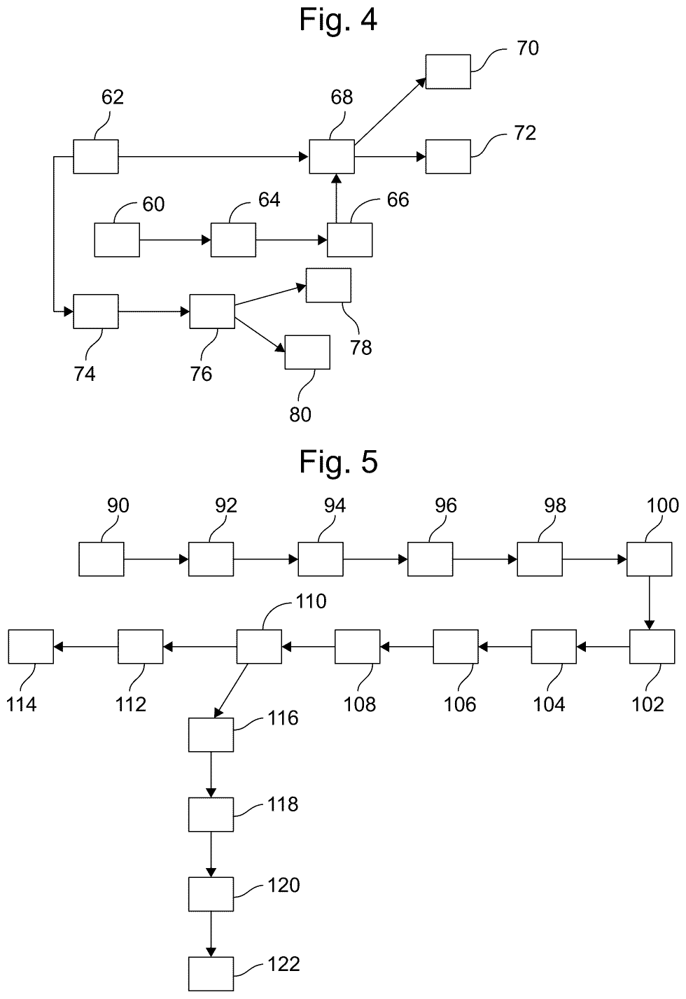

As shown in FIG. 4, the memory of the mobile computing device or the memory of the remote computer is configured for learning. During the learning phase, the sphygmomanometer 40 concomitantly measures 60 blood pressure as the wearable patch 12 is collecting 62 ultrasound data. The analogue to digital converter 32 of the sphygmomanometer 40 digitizes 64 the blood pressure data and the wireless radio 44 sends 66 the digitized blood pressure data to the mobile computing device 14. The two data sets are compared 68 and data from the wearable device 12 that do not agree with the data from the sphygmomanometer 40 are discarded 70. The process is repeated until the memory has a large enough set of valid data to be considered trained 72. In an alternative embodiment, the sphygmomanometer 40 is replaced with any other known and approved blood pressure monitor. In another alterative embodiment, the mobile computing device sends 74 the digitized blood pressure data to the remote computer. The two data sets are compared 76 and data from the wearable device 12 that do not agree with the data from the sphygmomanometer 40 are discarded 78. The process is repeated until the memory has a large enough set of valid data to be considered trained 80.

As shown in FIG. 5, a user releasably attaches 90 the wearable patch 12 to their skin, preferably on their arm after ensuring that the batteries are in the wearable patch and are charged. The user opens 92 the application on their mobile computing device 14 and clicks 94 on “start”. The mobile computing device 14 communicates 96 wirelessly to the microprocessor 24 to tell it to instruct the array of ultrasound transducers to function. The microprocessor instructs 98 the array of ultrasound transducers to begin emitting ultrasound waves. The ultrasound transducers 16 emit 100 ultrasound waves which are reflected 102 off the arterial walls. The ultrasound transducers 16 receive 104 the reflected ultrasound waves and send 106 ultrasound data to the high-speed analogue to digital converter 28, which converts 108 the ultrasound data to digitized data. The digitized data are then sent 110 wirelessly via the wireless radio 26 to the mobile computing device 14. The memory is configured to analyze the ultrasound data to determine the change in arterial wall diameter during the diastolic-systolic cycle, hence the ultrasound data are analyzed 112 and the blood pressure is displayed 114 on the screen of the mobile computing device 14 in real time. In an alternative embodiment, the mobile computing device sends 116 the digitized data wirelessly to the remote computer. The memory of the remote computer 15 is configured to analyze the ultrasound data to determine the change in arterial wall diameter during the diastolic-systolic cycle, hence the ultrasound data are analyzed 118 to provide blood pressure data. The blood pressure data is then sent 120 back to the mobile computing device 14 and the blood pressure is displayed 122 on the screen of the mobile computing device 14 in real time.

While example embodiments have been described in connection with what is presently considered to be an example of a possible most practical and/or suitable embodiment, it is to be understood that the descriptions are not to be limited to the disclosed embodiments, but on the contrary, is intended to cover various modifications and equivalent arrangements included within the spirit and scope of the example embodiment. Those skilled in the art will recognize or be able to ascertain using no more than routine experimentation, many equivalents to the specific example embodiments specifically described herein. Such equivalents are intended to be encompassed in the scope of the claims, if appended hereto or subsequently filed.

1. A blood pressure monitoring system, the blood pressure monitoring system comprising a wearable patch and a mobile computing device, the wearable patch including: a flexible housing which includes a contact surface; an adhesive on at least a part of the contact surface; an array of ultrasound transducers embedded in the flexible housing and facing the contact surface; a high-speed analogue to digital converter in electrical communication with the array of ultrasound transducers; and a microprocessor which includes a wireless radio for transmitting ultrasound data to the mobile computing device and is in electronic communication with the high speed analogue to digital converter, the mobile computing device including: a memory; a processor; a wireless radio; and a screen, wherein the memory is configured for machine learning and to instruct the processor to convert the ultrasound data into blood pressure data and to display the blood pressure data on the screen.

2. The blood pressure monitoring system of claim 1, wherein the flexible housing comprises silicon.

3. The blood pressure monitoring system of claim 2, wherein the flexible housing comprises a three dimensionally printed plastic polymer.

4. The blood pressure monitoring system of claim 3, wherein the high-speed analogue to digital converter is configured to operate at over one million samples per second.

5. A wearable blood pressure monitoring patch for use with a mobile computing device, the wearable blood pressure monitoring patch comprising: a flexible housing which includes a contact surface; an adhesive on at least a part of the contact surface; an array of ultrasound transducers embedded in the flexible housing and facing the contact surface; a high-speed analogue to digital converter in electrical communication with the array of ultrasound transducers; and a microprocessor which includes a wireless radio for transmitting ultrasound data to the mobile computing device and is in electronic communication with the high speed analogue to digital converter.

6. The wearable blood pressure monitoring patch of claim 5, wherein the flexible housing is a silicon housing.

7. The wearable blood pressure monitoring patch of claim 6, wherein the high speed analogue to digital converter is configured to operate at over one million samples per second.

8. A method of monitoring a user's blood pressure, the method comprising: a user selecting the blood pressure monitoring system of claim 1; the user providing the wearable patch with a power source; the user releasably attaching the wearable patch to a selected skin surface; the ultrasound transducer emitting ultrasound waves under control of the microprocessor; the ultrasound transducer receiving reflected ultrasound waves; the high-speed analogue to digital converter digitizing the reflected ultrasound waves to provide a data set; the wireless radio sending the data set to the mobile computing device; and the mobile computing device analyzing the data set to provide a blood pressure reading.

9. The method of claim 8 wherein the displaying the blood pressure reading is in real time.
